(19) United States
(12) Patent Application Publication
(10) Pub. No.: US 2022/0339224 A1
(43) Pub. Date: Oct. 27, 2022

(54) SEAWEED-BASED COMPOSITION

(71) Applicant: Cargill, incorporated, Wayzata, MN (US)

(72) Inventors: Guèba AGODA-TANDJAWA, Baupte (FR); Claudine LEFRANCOIS, Baupte (FR); Cindy LE GARNEC, Baupte (FR); Jacques Andrè Christian MAZOYER, Baupte (FR)

(73) Assignee: Cargill, Incorporated, Wayzata, MN (US)

(21) Appl. No.: 17/760,615

(22) PCT Filed: Sep. 15, 2020
(86) PCT No.: PCT/US2020/050897

§ 371 Date: Mar. 15, 2022

(30) Foreign Application Priority Data
Sep. 16, 2019 (EP) 19197487.2

(51) Int. Cl. A61K 36/04; A23L 17/60; A23L 33/105; A23K 10/30; A23C 11/10

(52) U.S. Cl. CPC A61K 36/04; A23L 17/60; A23L 33/105; A23K 10/30; A23C 11/103; A23V 2002/00

(57) ABSTRACT

The present invention relates to a seaweed-based composition comprising water, a seaweed powder and an additional component, said additional component being chosen from the group consisting of glucomannans, galactomannans, native starch and combinations thereof. Preferably, the red seaweed selected from the families of Gigartinaceae, Bangiophyceae, Palmariaceae, Hypneaceae, Cystocloniaceae, Solieriaceae, Phyllophoraceae and Furcellariaceae or combinations thereof.

### FIELD OF THE INVENTION

The present invention relates to a seaweed-based composition for use in food, beverages, nutritional products, dietary supplements, feed, personal care applications, pharmaceutical applications and industrial applications. The present invention also relates to a method for the manufacturing of the seaweed-based composition.

### BACKGROUND OF THE INVENTION

It is believed that the amount of seaweed production in the world is in the order of 20,000,000 t/year. Recently, improved ways of cultivating and harvesting of seaweeds were developed not only to increase production but also to enable a more efficient growth control. EP 2 230 895, EP 3 246 292 and WO 2017/131510 disclose examples of a cultivating system of seaweeds. However, in spite of recent developments in cultivating and harvesting seaweeds it is believed that the seaweeds produced still lack the versatility to be effectively used in a large range of applications.

Seaweeds are plant-like organisms that generally live attached to rock or other hard substrata in marine environments. Seaweeds may be microscopic such as microalgae but also enormous such as giant kelp that grows in “forests” and tower like underwater woods from their holdfasts at the bottom of the sea. Most of the seaweed species are either green (more than 6500 species), brown (about 2000 species), or red (about 7000 species) kinds.

Since hundreds of years, people recognized that seaweeds are beneficial for human as well as animal health and recently, various studies demonstrated that seaweeds are effective as fat substitutes. As people become more aware of the relation between diet and health, the consumption of seaweeds has been and is increasingly gaining attention. Nowadays, many new food products based on seaweeds have been developed and marketed, offering enhanced health benefits and the potential to decrease the risk of diseases. In addition to the vast health benefits when consumed directly or after minor pre-processing as dietary supplements, the seaweeds have a range of natural functional properties such as nutritional, physicochemical and textural properties; and when used as ingredients to manufacture various products, seaweeds may transfer to these products their advantageous functional properties.

For example, seaweeds exhibit water holding and rheological properties and have a natural ability to increase viscosity, form gels and/or act as emulsifiers. However, despite their excellent properties, seaweeds are far from being regarded as a commodity and this is mainly due to their low processing suitability. Most often, seaweeds are used as harvested, i.e. unprocessed, to modify or enhance various rheological properties of products manufactured therefrom. However, since freshly harvested seaweeds have a reduced shelf life, men used to dry them in order to extend their storage properties and grind or crush them into a powder, often called flour, in order to handle or pack the seaweeds more easily. US 2018/0000137 discloses a flour of crushed algae; WO 2008/050945, WO 2015/033331 and WO 2017/204617 disclose products such as bio-composites, personal care compositions and hard capsules made from grinded dry and wet seaweed; and several kinds of seaweed flours are available commercially and can even be ordered online.

One drawback of such processing is that a dried and crushed seaweed may lose its natural rheological properties. It was observed that the commercially available seaweed flours and those manufactured according to known processes may have a reduced ability to enhance viscosity and form gels.

There is therefore a need for a seaweed-based composition which has superior rheological properties. There is also a need for a natural, i.e. non-chemically modified, seaweed-based composition having optimal rheological properties.

### SUMMARY OF THE INVENTION

The present invention provides a seaweed-based composition (hereinafter the inventive composition) having improved functionality, i.e. having excellent rheological properties. In particular, the seaweed-based composition of the present invention has the capacity to influence the viscosity of products containing thereof. Also said composition may be able to produce a gel and can be used in amounts sufficiently low not to influence, or influence to a lesser extent, other properties of a product containing thereof.

It is therefore believed that the utilization of the inventive composition in the manufacturing of various products, imparts those products with excellent rheological properties and textures. In addition, the inventive composition may have a lesser impact on other properties of products containing thereof such as colour, taste, odour, mouthfeel, appearance and the like.

### EXPLANATION OF THE FIGURES

FIG. 1 shows the methodology to determine the C₀ of a seaweed powder sample.

FIG. 2 shows a mechanical spectra (variations of storage modulus (G′) and loss modulus (G″) as a function of frequency at 10° C.) of (a) a seaweed-based composition in accordance with the invention, containing Kappaphycus alvarezii powder and locust bean gum at a total concentration of 1% w/w with a mixing ratio of 50/50 prepared in distilled water and (b) of the corresponding individual components.

FIG. 3 shows a storage modulus (G′) and tan 6 (G″/G′) at 10° C. and 0.1 Hz obtained from the mechanical spectra of a seaweed-based composition in accordance with the invention, containing Kappaphycus alvarezii powder and locust bean gum at a total concentration of 1% w/w at a mixing ratio of 50/50 prepared in distilled water and of the corresponding individual components.

FIGS. 4a and 4b show the variation of the storage modulus (G′) and tan 6 (G″/G′) when varying the ratio of Kappaphycus alvarezii powder to locust bean gum.

FIG. 5 shows the mechanical spectra (variations of storage modulus (G′) and loss modulus (G″) as a function of frequency at 10° C.) of Kappaphycus alvarezii powder/Simpure 99400 native starch mixture at a mixing ratio of 0.3/2, and those of the individual component (0.3% w/w Kappaphycus alvarezii powder and 2% w/w Simpure 99400 native starch), taken as references, prepared in distilled water in presence of 0.3% KCl.

FIG. 6 shows the storage modulus (G′) and tan 6 (G″/G′) at 10° C. and 0.1 Hz obtained from the mechanical spectra of Kappaphycus alvarezii powder/Simpure 99400 native starch mixture at a mixing ratio of 0.3/2, and those of the individual component (0.3% w/w Kappaphycus alvarezii powder and 2% w/w Simpure 99400 native starch), taken as references, prepared in distilled water in presence of 0.3% KCl.

### DETAILED DESCRIPTION OF THE INVENTION

The invention relates to a seaweed-based composition comprising water, a seaweed powder and an additional component, said additional component being chosen from the group consisting of glucomannans, galactomannans, native starch and combinations thereof. Preferably, the additional component is chosen from the group consisting of guar gum, xanthan gum, locust bean gum, cassia gum, tara gum, konjac gum, alginate, agar, carrageenan, beta 1,3 glucan, native starch and combinations thereof. More preferably, the additional composition is chosen from the group consisting of guar gum, xanthan gum, locust bean gum, native starch and combinations thereof. Most preferably, the additional composition is chosen from the group consisting of locust bean gum and/or native starch.

Preferably, the additional component is used in an amount of at least 1.5 wt % based on the total dry solids content of the composition, more preferably at least 2.0 wt %, even more preferably at least 2.5 wt %, even more preferably at least 3.0 wt %, even more preferably at least 3.5 wt %, most preferably at least 4.0 wt %. Preferably, said amount is at most 90 wt %, more preferably at most 85 wt %, even more preferably at most 80 wt %, even more preferably at most 70 wt %, even more preferably at most 60 wt %, most preferably at most 55 wt %. Preferably, the additional component is used in an amount of between 1.5 wt % and 90 wt % based on the total dry substance amount contained by the composition, more preferably between 2.0 wt % and 80 wt %, even more preferably between 2.5 wt % and 70 wt %, even more preferably between 3.0 wt % and 65 wt %, even more preferably between 3.5 wt % and 60 wt %, most preferably between 4.0 wt % and 55 wt %.

As used herein, the term “dry solids” (DS) means the ratio of the weight of the solid content contained by a sample and the total weight of said sample. The solid content is herein understood the content of a sample obtained by evaporating the water contained by said sample by drying 5 g of the sample for 4 hours at 120° C. under vacuum (e.g. below 0.5 bar).

Preferably, the seaweed powder and the additional component are in a weight ratio of between 15.0:85.0 and 98.5:1.5, more preferably between 20.0:80.0 and 98.0:2.0, even more preferably between 30.0:70.0 and 97.5:2.5, even more preferably between 40:60 and 97.0:3.0, most preferably between 45.0:55.0 and 96.0:4.0.

Preferably, when the additional component is native starch (also referred to herein simply as starch), the starch is utilized in an amount of at least 5 wt % based on the total dry solids content of the composition, more preferably at least 20 wt %, even more preferably at least 40 wt %, most preferably at least 60 wt %. Preferably, the starch amount is at most 98 wt %, more preferably at most 94 wt %, even more preferably at most 90 wt %, most preferably at most 88 wt %. The native starch used in this invention may be any starch derived from any native source. A native starch is also a starch which is not chemically modified, i.e. it does not contain chemical compounds added via a chemical reaction. A native starch as used herein, is one as it is found in nature, whose properties may also be modified and/or enhanced by a physical treatment. Also suitable are starches derived from a plant obtained by any known breeding techniques. Typical sources for the native starches are cereals, tubers and holdfasts, legumes and fruits. The native source can be any variety, including without limitation, corn, potato, sweet potato, barley, wheat, rice, sago, amaranth, tapioca (cassava), arrow-holdfast, canna, pea, banana, oat, rye, triticale, and sorghum, as well as low amylose (waxy) and high amylose varieties thereof. Low amylose or waxy varieties is intended to mean a starch containing at most 10% amylose by weight, preferably at most 5%, more preferably at most 2% and most preferably at most 1% amylose by weight of the starch. High amylose varieties is intended to mean a starch which contains at least 30% amylose, preferably at least 50% amylose, more preferably at least 70% amylose, even more preferably at least 80% amylose, and most preferably at least 90% amylose, all by weight of the starch. The native starch may be physically treated by any method known in the art to mechanically and/or thermally alter the starch, such as by shearing or by changing the granular or crystalline nature of the starch, including conversion and/or pregelatinization. Methods of physical treatment known in the art include ball-milling, homogenization, high shear blending, high shear cooking such as jet cooking or in a homogenizer, drum drying, spray-drying, spray cooking, chilsonation, roll-milling and extrusion, and thermal treatments of low (e.g. at most 2 wt %) and high (above 2 wt %) moisture containing starch. As stated above, the native starch should not be chemically modified by treatment with any reagent or combination of reagents known in the art. Examples of chemical modifications include crosslinking, acetylation, organic esterification, organic etherification, hydroxyalkylation (including hydroxypropylation and hydroxyethylation), phosphorylation, inorganic esterification, ionic (cationic, anionic, nonionic, and zwitterionic) modification, succination and substituted succination of polysaccharides. Bleaching is not considered as chemical modification for the purpose of the invention. Such modifications are known in the art, for example in Modified starches: Properties and Uses. Ed. Wurzburg, CRC Press, Inc., Florida (1986).

The inventive composition also comprises water, preferably in an amount of at least 5 wt % based on the total weight of said composition, more preferably at least 10 wt %, even more preferably at least 15 wt %, most preferably at least 20 wt %. There is no upper limit to the amount of water contained by the inventive composition, for practical reasons, the amount of water is preferably at most 99 wt %.

The inventive composition may further comprise additives; preservatives; vitamins; sterols like phytosterols; antioxidants like polyphenols; beneficial minerals for human nutrition; whole vegetable extracts; cellulose such as microfibrillated cellulose and cellulose gel; dextrin; maltodextrin; sugars like sucrose, glucose; polyols like mannitol, erythritol, glycerol, sorbitol, xylitol, maltitol; protein or protein hydrolysate like plants or vegetables proteins and dairy proteins; oils and fat; surfactants; lecithin and combinations thereof. These substances can be used in amounts which can vary widely depending on the application of the composition, for most applications said amount being typically between 0.01 wt % and 50 wt % based on the total weight of the composition.

The inventive composition may also contain a salt; any salt soluble in water can be utilized. Non-limiting examples of salts include chloride salts, e.g. sodium chloride, potassium chloride, calcium chloride and ammonium chloride; sulphate salts, e.g. magnesium sulphate, iron sulphate, calcium sulphate, potassium sulphate, sodium sulphate; nitrate salts, e.g. calcium nitrate, sodium nitrate, potassium nitrate; phosphate salts, e.g. sodium phosphate, calcium phosphate, potassium phosphate; salts of organic acids and combinations thereof. Preferably, the salt is sodium chloride or potassium chloride. Most preferably, the salt used is a food grade salt, i.e. a salt as defined in the “Codex standard for food grade salt”, CX STAN 150-1985, Rev. 1-1997, Amend, 1-1999, Amend 2-2001. Using a salt has been found particularly beneficial when the additional component was native starch.

By seaweed powder is herein understood a collection of seaweed particles, i.e. said powder contains seaweed particles. Said particles may be obtained by crushing or milling a seaweed in wet or dry form or by processing the seaweed as detailed hereinbelow. Preferably, the seaweed particles have a D50 of preferably at least 20 μm, more preferably at least 50 μm, even more preferably at least 75 μm, even more preferably at least 85 μm, most preferably at least 120 μm. Preferably, said D50 is at most 750 μm, more preferably at most 500 μm, even more preferably at most 350 μm, most preferably at most 250 μm. Preferably, said D50 is between 20 μm and 750 μm, more preferably between 50 μm and 350 μm, most preferably between 75 μm and 250 μm.

Preferably, the seaweed particles have a D90 of preferably at least 125 μm, more preferably at least 100 μm, even more preferably at least 175 μm, most preferably at least 220 μm. Preferably, said D90 is at most 800 μm, more preferably at most 600 μm, most preferably at most 400 μm. Preferably, said D90 is between 125 μm and 800 μm, more preferably between 175 μm and 600 μm, most preferably between 220 μm and 400 μm.

Preferably, the seaweed particles have a D50 of at least 20 μm and a D90 of at least 125 μm, more preferably a D50 of at least 50 μm and a D90 of at least 175 μm, most preferably a D50 of at least 75 μm and a D90 of at least 220 μm.

Preferably, the seaweed powder utilized in the inventive composition contains before being added to said composition at least 80% dry basis of seaweed particles, more preferably at least 90% dry basis, even more preferably at least 92% dry basis, most preferably at least 96% wt % dry basis. The remaining wt % up to 100 wt % may contain foreign materials other than the seaweed particles which formed part of the biomass, e.g. algae, other strains of seaweed, etc.

The seaweed powder preferably has a storage modulus (G′) of at least 10 Pa as determined on a 0.3 wt % aqueous dispersion of said powder. Preferably, said powder has a critical gelling concentration (C₀) of at most 0.5 wt %, more preferably at most 0.3 wt %, most preferably at most 0.1 wt %. Preferably, said powder has a G′ of at least 15 Pa, more preferably at least 20 Pa, more preferably at least 30 Pa, more preferably at least 50 Pa, more preferably at least 70 Pa, more preferably at least 90 Pa, even more preferably at least 110 Pa, most preferably at least 120 Pa. Preferably, said G′ is at most 500 Pa, more preferably at most 400 Pa, even more preferably at most 300 Pa, most preferably at most 200 Pa.

The functionality of the seaweed powder can be varied within wide ranges depending on the type of seaweed used as the raw material in the production of said powder. For example, seaweed powders having a G′ value at least 30 Pa and even at least 50 Pa may be obtained from Spinosum, while higher G′ of at least 120 Pa and even at least 180 Pa, can be obtained from Chondrus or Cottonii, respectively.

The storage modulus G′ is commonly used to analyse the rheological properties of products, most often said products being used to make dispersions. G′ is a measure of a deformation energy stored in the dispersion during the application of shear forces and provides an excellent indication of the capability of said product to influence dispersion's viscoelastic behaviour. For the purpose of the invention, G′ was measured on an aqueous medium containing a reduced amount of 0.3 wt % of inventive powder relative to the total weight of the aqueous medium. It is highly desirable to achieve dispersions having G′ values as high as possible at powder concentrations as low as possible. Another indicator of the functionality of the seaweed powder is its critical gelling concentration (C₀). C₀ represents the lowest concentration of the seaweed powder in an aqueous medium below which no gel-like behaviour can be observed. C₀ is also referred to as the critical concentration of gelation and together with G′ are used to characterize the seaweed powder as presented in the METHODS OF MEASUREMENT section of the description.

By “aqueous dispersion” containing the inventive powder is herein understood a composition wherein said powder is dispersed in the aqueous medium, said aqueous medium preferably forming a continuous phase. Preferably, said powder is homogeneously dispersed in said medium. The powder may be dispersed inside the aqueous medium (i.e. in the bulk) but can also be present at any interface present in said aqueous medium, e.g. the interface between water and any component other than the powder, e.g. oil. Examples of dispersions include without limitation suspensions, emulsions, solutions and the like.

The term “aqueous medium” as used herein means a liquid medium which contains water, non-limiting example thereof including pure water, a water solution and a water suspension, but also aqueous liquid mediums such as those contained by dairy products, e.g. reconstituted skimmed milk, milk, yoghurt and the like; by personal care products such as lotions, creams, ointments and the like; and pharmaceutical products. Within the context of the present invention, most preferred aqueous medium for the determination of the G′ is reconstituted skimmed milk and therefore the G′ was measured on a solution of reconstituted skimmed milk containing 0.3 wt % of inventive powder relative to the total weight of the solution.

The seaweed powder has a C₀ of preferably at least 0.001 wt %, more preferably at least 0.005 wt %, even more preferably at least 0.010 wt %, most preferably at least 0.015 wt %. Preferably, said C₀ is at most 0.100 wt %, more preferably at most 0.095 wt %, more preferably at most 0.090 wt %, even more preferably at most 0.085 wt %, most preferably at most 0.080 wt %. Preferably, the C₀ is between 0.001 and 0.100 wt %, more preferably between 0.005 and 0.090 wt %, most preferably between 0.010 and 0.080 wt %. More preferably, C₀ is between 0.001 and 0.5 wt %, more preferably between 0.005 and 0.3 wt %, even more preferably between 0.010 and 0.1 wt %, most preferably between 0.010 and 0.08 wt %.

The seaweed powder has preferably a CIELAB L* value of at least 50, preferably at least 60, preferably at least 70, preferably at least 74, more preferably at least 76, even more preferably at least 78, most preferably at least 80. Preferably, the inventive powder has a CIELAB a* value of at most 5.0, more preferably at most 3.5, most preferably at most 2.0. Preferably, the inventive powder has a CIELAB b* value of at most 20, more preferably at most 17, most preferably at most 15. Such values of L*, a* and b* ensure that the seaweed powder will interfere less with the desired colour of the products in which it is used, i.e. the seaweed powder is colour-neutral.

Preferably, the seaweed powder has a Cl⁻ content of at most 20 wt % relative to the weight of the powder, more preferably at most 15 wt %, even more preferably at most 10 wt %, most preferably at most 5 wt %. Preferably, said Cl⁻ content is at least 0.01 wt %, more preferably at least 0.1 wt %, most preferably at least 1 wt %. Such values of Cl⁻ content ensure that the seaweed powder will interfere less with the taste of the products in which it is used, i.e. the seaweed powder is taste-neutral.

Preferably, the seaweed powder contains an amount of acid insoluble material (AIM) of at most 50 wt % relative to the weight of the powder, more preferably at most 40 wt %, even more preferably at most 30 wt %, most preferably at most 20 wt %. Preferably, said AIM content is at least 1 wt %, more preferably at least 5 wt %, most preferably at least 10 wt %. It was observed that when the seaweed powder has an AIM content within the preferred ranges, it's nutritional properties were optimized.

Preferably, the seaweed powder contains an amount of acid insoluble ashes (AIA) of at most 5.0 wt % relative to the weight of the powder, more preferably at most 3.0 wt %, even more preferably at most 1.0 wt %, most preferably at most 0.80 wt %. Preferably, said AIA content is at least 0.01 wt %, more preferably at least 0.05 wt %, most preferably at least 0.10 wt %. It was observed that a seaweed powder having an AIA content within the preferred ranges, is more suitable for use in food, personal care and pharmaceutical products as it does not introduce, or introduce to a lesser extent, foreign materials into said products, which in turn may require additional purification steps of said products.

The seaweed suitable for the present invention may be selected from numerous types of seaweeds. In the present context by “seaweed” is understood a macroscopic, multicellular, marine algae which can grow in the wild or can be farmed. Wild seaweeds typically grow in the benthic region of the sea or ocean without cultivation or care from humans. Farmed seaweeds are typically cultivated on various supports like ropes, fabrics, nets, tube-nets, etc., which are typically placed below the surface of the sea or ocean. Seaweeds may also be farmed in pools, ponds, tanks or reactors containing seawater and placed on the shore or inland. The term “seaweed” includes members of the red, brown and green seaweeds.

Throughout this document, certain taxonomies of seaweeds' families, genera, etc. are used. The referred taxonomies are those typically used in the art of seaweed cultivation and harvesting and/or in the art of seaweed extracts. An explanation of the taxonomies of red seaweeds are for example given by C. W. Schneider and M. J. Wynne in Botanica Marina 50 (2007): 197-249; by G. W. Sauders and M. H. Hommersand in American Journal of Botany 91(10): 1494-1507, 2004; and by Athanasiadis, A. in Bocconea 16(1): 193-198.2003.—ISSN 11204060. An explanation of the taxonomies of green seaweeds is for example given by Naselli-Flores L and Barone R. (2009) Green Algae. In: Gene E. Likens, (Editor) Encyclopedia of Inland Waters. volume 1, pp. 166-173 Oxford: Elsevier. An explanation of the taxonomies of brown seaweeds is for example given by John D. Wehr in Freshwater Algae of North America—Ecology and Classification, Edition: 1, Chapter: 22, Publisher: Academic Press, Editors: John D. Wehr, Robert G. Sheath, pp. 757-773.

The seaweed used in accordance with the invention comprises a red seaweed, i.e. a seaweed belonging to Rhodophyta phylum; more preferably, said seaweed is a red seaweed. In addition to the red seaweed, the seaweed may also comprise a brown seaweed, i.e. orders, families and genera in the class Phaeophycaeae. Red seaweeds have a characteristic red or purplish colour imparted by pigments present in the seaweed and called phycobilin, e.g. phycoerythrin.

The invention also relates to a seaweed-based composition comprising water, a seaweed powder and an additional component, said component being chosen from the group consisting of glucomannans, galactomannans, native starch and combinations thereof, wherein the seaweed is belonging to Rhodophyta phylum. Preferably, the additional component is chosen from the group consisting of guar gum, xanthan gum, locust bean gum, cassia gum, tara gum, konjac gum, alginate, agar, carrageenan, beta 1,3 glucan, native starch and combinations thereof. More preferably, the additional composition is chosen from the group consisting of guar gum, xanthan gum, locust bean gum, native starch and combinations thereof. Most preferably, the additional composition is chosen from the group consisting of locust bean gum and/or native starch. More preferably, the seaweed is a red seaweed selected from the families of Gigartinaceae, Bangiophyceae, Palmariaceae, Hypneaceae, Cystocloniaceae, Solieriaceae, Phyllophoraceae and Furcellariaceae or combinations thereof. Most preferably, the seaweed is selected from the genera of Bangiales, Chondrus, Iridaea, Palmaria, Gigartina, Gracilaria, Gelidium, Rhodoglossum, Hypnea, Eucheuma, Kappaphycus, Agarchiella, Gymnogongrus, Sarcothalia, Phyllophora, Ahnfeltia, Mazzaella, Mastocarpus, Chondracanthus, Furcellaria and mixtures thereof. Best results were obtained when the seaweed was chosen from the group of seaweeds consisting of Porphyra sp., Palmaria palmata, Eucheuma spinosum, Eucheuma denticulatum, Eucheuma sp., Eucheuma cottonii (also known as Kappaphycus alvarezii), Kappaphycus striatus, Kappaphycus sp., Chondrus crispus, Irish moss, Fucus crispus, Chondrus sp, Sarcothalia crispata, Mazzaella laminaroides, Mazzaella sp., Chondracanthus acicularis, Chondracanthus chamissoi, Chondracanthus sp., Gigartina pistilla, Gigartina mammillosa, Gigartina skottsbergii, Gigartina sp., Gracilaria sp, Gelidium sp., Mastocarpus stellatus and mixtures thereof.

It is known that some of the red seaweeds, e.g. Kappaphycus alvarezii, may have green or brown strains; however, within the context of the present invention when mentioning for example that the seaweed is a red seaweed, it is herein meant the phylum and not the colour of the strains.

Most preferred brown seaweeds are those chosen from the families Acsophyllum, Durvillaea, Ecklonia, Hyperborea, Laminaria, Lessonia, Macrocystis, Fucus and Sargassum. Specific examples of brown seaweeds include Bull Kelp (Durvillae potatorum), Durvillae species, D. antarctica and Knotted Kelp (Ascophyllum nosodum).

Preferably, the seaweed powder has a storage modulus (G′) of at least 10 Pa as determined on a 0.3 wt % aqueous dispersion of said powder and a critical gelling concentration (C₀) of at most 0.5 wt %, wherein the seaweed is a red seaweed, i.e. a seaweed belonging to Rhodophyta phylum. Preferred ranges of the G′ and C₀ are given above and will not be repeated herein. Preferably, said powder has a CIELAB L* value of at least 50, preferably at least 60, preferably at least 70, preferably at least 74, more preferably at least 76, even more preferably at least 78, most preferably at least 80. Preferably, the seaweed is a red seaweed selected from the families of Gigartinaceae, Bangiophyceae, Palmariaceae, Hypneaceae, Cystocloniaceae, Solieriaceae, Phyllophoraceae and Furcellariaceae or combinations thereof. Most preferably, the seaweed is selected from the genera of Bangiales, Chondrus, Iridaea, Palmaria, Gigartina, Gracilaria, Gelidium, Rhodoglossum, Hypnea, Eucheuma, Kappaphycus, Agarchiella, Gymnogongrus, Sarcothalia, Phyllophora, Ahnfeltia, Mazzaella, Mastocarpus, Chondracanthus, Furcellaria and mixtures thereof.

Most preferably, the seaweed powder has a storage modulus (G′) of at least 10 Pa as determined on a 0.3 wt % aqueous dispersion of said powder and a critical gelling concentration (C₀) of at most 0.5 wt %, wherein the seaweed is a red seaweed chosen from the group consisting of Eucheuma spinosum, Eucheuma Cottonii (Kappaphycus alvarezii), Chondrus crispus and combinations thereof. Preferred ranges of the G′ and C₀ are given above and will not be repeated herein. Preferably, said powder has a CIELAB L* value of at least 50, preferably at least 60, preferably at least 70, preferably at least 74, more preferably at least 76, even more preferably at least 78, most preferably at least 80.

The inventive composition can be manufactured according to any known method such as for example known mixing or blending methods. In one embodiment, the inventive composition is manufactured by a process comprising:

a) Providing the seaweed powder and the additional component;

b) Mixing the seaweed powder with the additional component;

c) Adding water before, during or after step b).

Embodiments and amounts of the seaweed powder and of the additional component are presented hereinabove and will not be repeated herein.

Any mixing methods can be used, e.g. using a blender or any known missing device. Water can be added to the individual components, during the mixing of the components or after the components have been mixed. By water is herein understood any aqueous solution containing water, e.g. milk, skimmed milk, syrup, etc.

Preferably, the seaweed powder is manufactured by a process comprising the steps of:
a) Providing a biomass containing seaweed and water, and having a dry solids (DS) content of at least 5 wt %.
b) Subjecting the biomass to an exudation process to exude the water present inside the seaweed and obtaining an exudated biomass containing an exudated seaweed;
c) Optionally drying the exudated biomass to a moisture level of at most 40 wt % to obtain a dried, exudated biomass;
d) Cooking the exudated biomass in a brine solution to obtain a cooked biomass;
e) Optionally washing and/or drying the cooked biomass; and
f) Transforming the cooked biomass of step d) or e) into a seaweed powder.

It is preferred that the inventive method uses live seaweed largely unaffected by decomposition and/or fermentation. It is therefore highly desirable that the inventive method does not involve fermentation of the seaweed, i.e. it is a non-fermentative method.

In step a) of the inventive method, all parts of the seaweed can be used, e.g. holdfasts, stem and leaves, for making the biomass. The seaweed can be used whole, cut or otherwise mechanically manipulated.

Preferably, at step a) the biomass contains a cleaned seaweed and has a DS of at least 15 wt %, more preferably at least 30 wt %, most preferably at least 55 wt %. Preferably, the DS is at most 95 wt %, more preferably at most 85 wt %, most preferably at most 80 wt %. Preferably, said DS is between 5 and 95 wt %, more preferably between 30 and 85 wt %, most preferably between 55 and 80 wt %.

It is essential to carry out step b) of the inventive method on a biomass containing a seaweed capable of exuding. The process at step b) aims to exude in a carefully controlled environment the water present inside the seaweed (the water internal to the seaweed also known as the water of hydration of the seaweed), i.e. inside the holdfasts, stems and leaves thereof.

Preferably, step b) utilizes a live biomass, i.e. a biomass which was not dried between the harvest of the seaweed and the commencing of the exudation step. By “live, harvested” seaweed is herein understood a seaweed that is kept alive after harvest, has biological activity such as respiration and has the ability to exude. In clear distinction to live, harvested seaweed, dried seaweed is dead, has no biological activity such as respiration and is no longer capable of exudation. A dead seaweed may be rehydrated with water to some extent and in this case may be able to exude some of that water, however, utilizing rehydrated dead seaweed in step b) of the inventive method is less preferred. Preferably, the biomass is also fresh.

To ensure that the biomass utilized at step b) of the inventive method is a live and fresh biomass, preferably step b) takes place within 15 days from harvesting the seaweed, more preferably within 2 days from the harvest, even more preferably within 24 hours from the harvest, most preferably within 4 hours from the harvest. After carrying out step b), the exudated seaweed may still be recognizable as seaweed botanically and taxonomically as the exudation process used in the inventive method is a natural process.

The exudation process at step b) preferably takes place under carefully adjusted conditions, e.g. in an environment (hereinafter the “exudation environment”) containing at least 50 wt % moisture, more preferably at least 70 wt % moisture, even more preferably at least 80 wt % moisture, even more preferably at least 90 wt % moisture, most preferably at least 95 wt % moisture. To reach the high moisture content of the exudation environment, water, preferable seawater, can be added or sprinkled onto the seaweed or inside the exudation environment.

Preferably, the exudation is carried out at an exudation temperature of at least 20° C., more preferably of at least 30° C., even more preferably of at least 40° C., yet even more preferably at least 50° C., yet even more preferably at least 60° C., most preferably at least 70° C. Preferably, said temperature is at most 150° C., more preferably at most 120° C., most preferably at most 90° C. Preferably, said temperature is between 40 and 150° C., more preferably between 50 and 120° C., most preferably between 60 and 90° C. Using such temperatures, guarantees an optimum exudation process.

The typical duration for exudation will vary by species, season of harvest, quantity of moisture present in the exudation environment and exudation temperature. Generally, the period of exudation will be at least 3 hours, preferably at least 8 hours, more preferably at least 12 hours, most preferably at least 24 hours. Preferably, the exudation period will be between 3 hours and 10 days, more preferably between 8 hours and 4 days, most preferably between 12 hours and 2 days.

The exudation process results in a biomass containing an exudated seaweed, i.e. a seaweed which is desiccated or dehydrated. Preferably said process is carried out to extract at least 5 wt % of the water present inside the seaweed, more preferably at least 10 wt %, most preferably at least 15 wt %. Preferably, the amount of extracted water is at most 50 wt % of the water present inside the seaweed, more preferably at most 30 wt %, most preferably at most 20 wt %. The amount of water inside the seaweed can be determined by taking samples of the seaweed at certain time intervals, and weighing the seaweed before and after drying it at a temperature of 120° C. until no weight change occurs.

Preferably, the environment in which the exudation step is carried out is a closed environment, i.e. an environment wherein the flow of air is preferably below 1 m/s.

The exudated biomass may be subjected to an optional drying process before being cooked in brine solution. The drying step preferably decreases the amount of moisture contained by the exudated biomass to at most 40 wt % based on the weight of the biomass. Preferably, the moisture content of the dried biomass is at most 35 wt %, even more preferably at most 30 wt %, most preferably at most 25 wt %. Preferably, the moisture content of said dried biomass is at least 5 wt %, more preferably at least 10 wt %, most preferably at least 15 wt %. Drying the exudated biomass before cooking, may allow an easier manipulation thereof.

After the optional drying and before being cooked, the exudated biomass is preferably rehydrated by the addition of water, which can be fresh or seaweed water. The rehydration preferably results in a rehydrated biomass having a DS of at least 20 wt %, more preferably at least 30 wt %, most preferably at least 40 wt %. Preferably, the DS is at most 80 wt %, more preferably at most 70 wt %, most preferably at most 60 wt %. Preferably, said DS is between 20 and 80 wt %, more preferably between 30 and 70 wt %, most preferably between 40 and 60 wt %.

Any drying method can be used to reduce the moisture content of the biomass. An advantageous drying method is low temperature drying using dehumidified air. Such drying method have the ability to preserve heat sensitive compounds of the seaweed such as proteins, fibers, starch and other nutrients and hence retaining seaweed quality. Other techniques may include ventilated chamber drying, oven drying, sun drying, (forced-flow) evaporation, flash drying, zeolite drying, fluidized bed drying, and the like.

The exudated biomass (whether or not dried and rehydrated), is subsequently cooked in a brine solution to obtain a cooked biomass. The brine is an aqueous solution containing at least one salt and having a salt concentration at room temperature (20° C.) of preferably at least 3 wt % relative to the total weight of the solution. Preferably, the concentration of salts is at least 5 wt %, more preferably at least 7 wt %, most preferably at least 10 wt %. Preferably, said salt concentration is at most 50 wt %, more preferably at most 40 wt %, most preferably at most 30 wt %.

The cooking preferably takes place at a cooking temperature of at least 85° C., more preferably at least 86° C., even more preferably at least 88° C., most preferably at least 90° C. Preferably, the cooking temperature is at most 100° C., more preferably at most 98° C., even more preferably at most 96° C., most preferably at most 95° C. Preferably, the cooking temperature is between 85 and 100° C., more preferably between 86 and 96° C., even more preferably between 88 and 96° C., most preferably between 90 and 95° C.

The cooking time is preferably at least 25 minutes, more preferably at least 30 minutes most preferably at least 35 min. Preferably, the cooking time is at most 60 min, more preferably at most 55 min, even more preferably at most 50 min, most preferably at most 40 min Preferably, the cooking time is between 25 and 60 min, more preferably between 30 and 55 min, even more preferably between 30 and 50 min, most preferably between 30 and 40 min.

The cooking step can be carried out by contacting the biomass with brine solution in a bath of said solution or a succession of baths of said solution. During the cooking process, preferably enough brine solution is used to cover the seaweed entirely. Preferably care is taken to prevent evaporation of the brine solution and a change in the salt concentration, e.g. by carrying out the cooking in a closed vessel. Alternatively, water can be added during the cooking to prevent the seaweed from being exposed to air.

A brine solution which has been found to be very effective for the purpose of the invention is a solution of sodium or potassium chloride. It is however understood that any salts other than sodium or potassium chloride can be used, non-limiting examples including other chloride salts, sulphates, nitrates, carbonates, phosphates, salts of organic acids and combinations thereof. The only qualification is that the salt should be sufficiently soluble to permit the formation of a brine solution at the required concentrations. It is also preferred that the salt is not excessively acidic nor basic in its reaction, namely, in aqueous solution, the pH of the solution is preferably between 6.0 and 10.0. If the produced seaweed powder is intended for being utilized in food, feed, personal care or pharma products, preferably the salt is a salt whose presence is allowed in such products.

The inventors observed that such careful cooking process may prevent the degradation of the seaweed and help in producing powders with excellent properties. Even more surprisingly, the inventors observed that the cooking step may improve the dispersability of the inventive composition. It was observed that the inventive composition can be homogeneously dispersed inside an aqueous medium without the occurrence of lumps or particulates whereas non-cooked seaweeds produced visible particulates. After cooking, the biomass may be subjected to a filtration step to remove the water before the subsequent washing step. The filtration can be accomplished by any suitable type of equipment of which many are well known, e.g. filter press cylinder type filter, or the like. If desired, a centrifugal machine can also be used.

According to step e) of the inventive process, the biomass is washed. Any washing method can be used as for example rinsing under a flow of water, placing the biomass in a volume of water and combinations thereof. The washing may be carried out in one or several water baths, in a tank provided with suitable agitator means or in any washing system such as batch or continuous systems, in co- or counter-current configurations. Good results were obtained when the biomass was rinsed several times with fresh water.

Before being dried, the washed biomass can be subjected to a filtration step to remove the water therefrom and aid drying. The filtration can be accomplished by any suitable type of equipment of which many are well known, e.g. filter press, cylinder type filter, presses, sieves, or the like. If desired, a centrifugal machine can also be used.

The washed biomass is dried to a moisture content suitable to permit mechanical manipulation of said biomass. Any types of driers can be used, like vacuum driers, drums driers, air lift driers, etc. Preferably, the moisture content of the dried biomass is at most 25 wt %, more preferably at most 20 wt %, even more preferably at most 15 wt %, most preferably at most 12 wt %. Preferably, the moisture content of said biomass is at least 4 wt %, more preferably at least 6 wt %, even more preferably at least 8 wt %, most preferably at least 10 wt %. Preferably, said moisture content is at most 20 wt %, more preferably at most 15 wt %, most preferably at most 12 wt %. Preferably, said moisture content is between 4 wt % and 20 wt %, more preferably between 6 wt % and 15 wt %, most preferably between 8 wt % and 12 wt %.

The dried biomass may be transformed into a seaweed powder by using a mechanical treatment. Mechanical treatments include for example cutting, milling, pressing, grinding, shearing and chopping. Milling may include for example, ball milling, hammer milling, conical or cone milling, disk milling, edge milling, rotor/stator dry or wet milling, or other types of milling. Other mechanical treatments may include stone grinding, cracking, mechanical ripping or tearing, pin grinding, burr grinding, or air attrition milling. The mechanical treatment can be configured to produce powders with specific morphology characteristics such as for example, surface area, porosity, bulk density, and in case of fibrous seaweed, fibre characteristics such as length-to-width ratio.

If desired, the obtained powder can be passed through a screen, e.g. having an average opening size of 0.25 mm or less.

The biomass may at any step during the process, but preferably after step b), be subjected to a sterilisation step to reduce the microbiota thereof and/or eliminate the harmful species. It is known that the surface of seaweeds supports a diverse microbiota (such as fungi, bacteria, viruses, spore forms, etc.), generally within biofilms, some species being harmful to humans, e.g. Escherichia coli and Enterococcus. Sterilisation may be achieved by applying the proper combination of heat, irradiation, high pressure and filtration. Heat treatments in the presence or absence of water are known to reduce the microbial levels. For example, a treatment of seaweed for at least 10 minutes at 121° C. in a humid environment is known to ensure sterility. Other sterilisation methods including irradiation with gamma rays or microwaves, ozone treatment, pulsed light treatment, disinfection with alcohol and combinations thereof may be used.

The inventors observed that the inventive composition may positively influence mixing, sheeting, extrusion, baking, frying, and roasting characteristics of human and animal food; may be used to advantageously modify the rheology of sauces, dips, beverages, soups and other liquid, semi-liquid and/or semi-solid products; may be used to provide products with interesting textures, good appearance and the like.

The present invention also relates to a dietary blend comprising the inventive composition and a therapeutic agent such as an absorption altering agent, an appetite altering agent, a metabolism altering agent, a cholesterol altering agent or any combination thereof. Examples of such agents are given in WO 2016/085322, the disclosure thereof being incorporated herein by reference.

The present invention also relates to a pharmaceutical blend comprising the inventive composition and a pharmaceutically acceptable carrier and/or an excipient and/or a diluent. The excipient/diluent/carrier(s) must be “acceptable” in the sense of being compatible with the therapeutic agent and not deleterious to the recipients thereof.

The inventive composition may form part of a food product, beverage, nutritional product, dietary supplement, feed product, personal care product, pharmaceutical product or industrial product. Thus, an aspect of the invention relates to a food product, a beverage, a nutritional product, a dietary supplement, a feed product, a personal care product, a pharmaceutical product or industrial product comprising the inventive composition. In the present context “food” refers to eatable material suitable for human consumption, whereas feed refers to eatable material suitable for animal consumption. The food or feed ingredient may also form part of a food or feed product.

The invention further relates to a food or a feed product containing the inventive composition and a nutrient. Without being bound to any theory, the inventors believe that the dynamics and kinetics of the nutrient uptake by the one ingesting said food or feed product may be positively influenced by the advantageous properties of the inventive composition. In particular the inventive composition may enable an optimization of the transport, diffusion, and dissolution phenomena relevant to food functionalities (nutritional, sensory, and physicochemical). Moreover, said products may be easily designed to have specific flow behaviors, textures and appearances. Thus, the ability of the inventive composition to optimize said food functionalities may be highly beneficial for the design of food structure, which together with the classic needs (e.g. texture and mouthfeel), may enhance the impact upon wellness and health, including modulated digestion to trigger different physiological responses.

The inventive composition is suitably used in the production of a large variety of food compositions. Examples of food compositions comprising thereof, to which the invention relates, include: luxury drinks, such as coffee, black tea, powdered green tea, cocoa, adzuki-bean soup, juice, soya-bean juice, etc.; milk component-containing drinks, such as raw milk, processed milk, lactic acid beverages, etc.; a variety of drinks including nutrition-enriched drinks, such as calcium-fortified drinks and the like and dietary fibre-containing drinks, etc.; dairy products, such as butter, cheese, yogurt, coffee whitener, whipping cream, custard cream, custard pudding, etc.; iced products such as ice cream, soft cream, lacto-ice, ice milk, sherbet, frozen yogurt, etc.; processed fat food products, such as mayonnaise, margarine, spread, shortening, etc.; soups; stews; seasonings such as sauce, TARE, (seasoning sauce), dressings, etc.; a variety of paste condiments represented by kneaded mustard; a variety of fillings typified by jam and flour paste; a variety or gel or paste-like food products including red bean-jam, jelly, and foods for swallowing impaired people; food products containing cereals as the main component, such as bread, noodles, pasta, pizza pie, corn flake, etc.; Japanese, US and European cakes, such as candy, cookie, biscuit, hot cake, chocolate, rice cake, etc.; kneaded marine products represented by a boiled fish cake, a fish cake, etc.; live-stock products represented by ham, sausage, hamburger steak, etc.; daily dishes such as cream croquette, paste for Chinese foods, gratin, dumpling, etc.; foods of delicate flavour, such as salted fish guts, a vegetable pickled in sake lee, etc.; liquid diets such as tube feeding liquid food, etc.; supplements; and pet foods; creamers (dairy and non-dairy), condensed milk, alcoholic beverages, in particular those containing dairy products, e.g. Irish cream whiskey and the like; and sport drinks. These food products are all encompassed within the present invention, regardless of any difference in their forms and processing operation at the time of preparation, as seen in retort foods, frozen foods, microwave foods, etc. Due to its neutral taste and odour, such food products are not, or are less, affected by the natural taste or smell of the seaweed.

The present invention further relates to the use of the inventive composition in dairy products, e.g. yogurt {e.g., spoonable, drinkable, and frozen), sour cream, cheese products, sauces (cheese and white), pudding, and frozen desserts. Unexpectedly, it was observed that the inventive composition can be used in dairy products with a resulting smooth texture and essentially without any loss in viscosity or creaminess. Said inventive composition can be used as an ingredient or as an additive to dairy products, i.e. in addition to the fat contained by such products. Alternatively, said inventive composition can be used to substitute some or even all of the fat in dairy products, to obtain reduced-fat or fat-free products in which case such use may result in a decreased caloric content of the final dairy product {e.g., a reduction of at least 10%, or at least 50%).

As used herein, additive means any substance added to a base material in low concentrations for a definite purpose. In the United States, the Food and Drug Administration sets the allowable levels of food additives after evaluating the safety and toxicity of the additive. Additives may be essential to the existence of the end product, such as the use of emulsifiers in mayonnaise or leavening agents in bread products. Alternatively, additives may perform a secondary function, e.g. may function as thickeners, flavouring agents, or colouring agents. The inventive composition described herein may be used as additive in dairy products but also as an ingredient.

Dairy product as used herein means milk or any food product prepared from non-vegetable milk (e.g., cow milk, sheep milk, goat milk, and the like), whether in a dry or a non-dry form, including butter, cheese, ice cream, pudding, sour cream, yogurt (e.g., spoonable, drinkable, and frozen) and condensed milk. In a less preferred embodiment, products manufactured with vegetable milk, e.g. soy milk, and vegetable milk-based products can also be used in the examples described herein.

Cheese is herein understood as a food prepared from the pressed curd of milk, often seasoned and aged.

Lipid is a term describing a product comprising fats and/or fat-derived materials. Fat is herein understood as an ester of glycerol and three fatty acids. A fatty acid is a carboxylic acid typically having a carbon chain from 4-22 carbon atoms in length and usually having an even number of carbon atoms in the chain. The fatty acids can be saturated, i.e., containing no double bonds, or unsaturated, i.e., containing one or more double bonds. Fats can be found both in animal products and in some plant products.

Ice cream is herein understood as a smooth, sweet, cold food prepared from a frozen mixture of milk products and flavourings. In the United States ice cream contains a minimum of 10% milkfat and 10% non-fat milk solids (see, 2 1 C.F.R. § 135.1 10). However, the disclosure is not limited to this specific range, as the required percentages of milkfat and non-fat milk solids in ice creams can vary in other countries or jurisdictions.

Yogurt is herein understood as a dairy product produced by culturing cream, milk, partially skimmed milk, or skim milk with a characterizing bacterial culture that contains lactic acid-producing bacteria, such as Lactobacillus delbrueckii ssp. and Streptococcus thermophilus. Exemplary yogurts include, but are not limited to, spoonable yogurt, yogurt dip, frozen yogurt, and drinkable yogurt. By definition in 2 1 C.F.R. § 13 1.200, regular yogurt in the United States has a milkfat content of at least 3.25%. The fat content of regular yogurts typically ranges from 3.25% to about 3.8%, although there are yogurts on the market with a fat content of about 10%. As defined in 21 C.F.R. § 13 1.203, in the United States low-fat yogurts have not less than 0.5% milkfat and not more than 2% milkfat. A non-fat yogurt has less than 0.5% milkfat in the United States as defined in 2 1 C.F.R. § 131.206. However, other ranges maybe observed in other countries.

Dairy products may be prepared using methods known to those skilled in the art, e.g. WO2009/079002, except that the inventive composition is added or used to replace some or all of the fat in said products. Said inventive composition can be added at one of several points during the manufacture of the dairy product, e.g. they may be added to the milk prior to pasteurization. Said inventive composition can be added in its dry form or, alternatively, an aqueous dispersion may be prepared by dispersing said inventive composition in an aqueous environment and then adding said dispersion to the milk.

The inventive composition can be used to substitute some or all of the fat in the dairy product. Preferably, said inventive composition are used in an amount sufficient to substitute at least 5% of the fat, more preferably said amount substitutes at least 10% of said fat, even more preferably at least 20%, yet more preferably at least 50%, yet more preferably at least 75%, most preferably essentially all fat is replaced by said inventive composition.

The inventive composition is preferably added to the dairy product in an amount of up to 10 wt % relative to the weight of the product, more preferably up to 7 wt %, even more preferably up to 5 wt %, most preferably up to 3 wt %. Preferably said amount is between 0.01 and 10 wt %, more preferably between 0.03 and 7 wt %, most preferably between 0.05 and 5 wt %.

The inventive composition may also be used in cosmetic formulations. The invention therefore relates to a cosmetic formulation comprising said composition. Non-limiting examples of cosmetic formulations include basic cosmetics (facial toilet, milks, creams, ointments, lotions, oils and packs), facial washes, skin washes, hair cosmetics such as shampoo, rinse and the like, and makeup cosmetics such as lipstick, foundation, blush, eye shadow, mascara, and the like.

The inventive composition may also be utilized into bath salts, tooth paste, deodorizers, sanitary cottons, wet tissues, and the like. The invention therefore also relates to such products containing said composition.

The inventive composition may also be used in the manufacturing of industrial products, e.g. sealants, adhesives, paper, and other building materials.

Any feature of a particular embodiment of the present invention may be utilized in any other embodiment of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the examples and comparative experiments, or where otherwise explicitly indicated, all numbers in this description indicating amounts of material or conditions of reaction, physical properties of materials and/or use are to be understood as modified by the word “about”. Unless specified otherwise, numerical ranges expressed in the format “from x to y” are understood to include x and y. When for a specific feature multiple preferred ranges are described in the format “from x to y”, it is understood that all ranges combining the different endpoints are also contemplated. For the purpose of the invention ambient (or room) temperature is defined as a temperature of about 20 degrees Celsius.

### Methods of Measurement

Cl⁻ amount was measured by potentiometric titration (Metrohm) with AgNO₃. 200 to 300 mg of the sample (Wₛₐₘₚₗₑ) were added to 150 ml osmosis water in a 250 ml beaker. The sample was stirred until a homogeneous dispersion of the sample was achieved. 4 to 5 drops of fuming nitric acid were added to the sample. The titration was carried out with a potentiometer (682 Titroprocessor, Metrohm) and a combined electrode Ag/AgNO₃. The wt % of chlorides can be directly calculated with the formula: % Cl⁻=V×C×M[Cl]×100/Wₛₐₘₚₗₑ with M[Cl] being 35.5 g/mol and where V is the volume of AgNO₃ (in mL) solution utilized and C is its concentration, i.e. 0.1N.
AIM was measured by dispersing 0.5 g of sample (Wₛₐₘₚₗₑ) in 150 ml osmosis water in a 250 mL beaker. 1.5 mL of concentrated sulfuric acid were added thereto. The beaker was covered with plastic foil to prevent evaporation and heated on bain-marie at boiling temperature for 2 h. The dispersion was centrifuged at 4000 rpm (equivalent to 3250 g) for 10 minutes.
The total mass (W_{filter+dish}) of a AP 25 filter and a crystallizing dish was determined. The acidic dispersion was filtered and rinsed with osmosis water at 50° C. until its pH remained neutral (as check with a pH paper)—about 500 mL water were used. The filter with the sample was allowed to dry overnight at room temperature and further dried in an oven at 60° C. for a day and the total weight of the sample, filter and dish was determined (W_{final}) AIM=[(W_{final}−W_{filter+dish})/Wₛₐₘₚₗₑ]×100.
AIA was measured as follows: 2,000 (two) grams (Wₛₐₘₚₗₑ) of sample were placed on a silica or platinum crucible, burnt for about one hour on a hot plate at 500° C. and subsequently placed in a furnace at 550° C. for 16 h. The obtained ashes were added to a solution containing 10 ml concentrated HCl and 20 ml demineralized water. The solution containing the ashes was heated to 80° C. for about half an hour and subsequently filtered using a Whatman No 40 (ash free filter). The filter containing the ashes was rinsed with water until no Cl⁻ were detected in the sample. The presence of Cl⁻ in the sample was checked with AgNO₃ (the precipitation of AgCl signifies the presence of Cl⁻).

A second silica or platinum crucible was placed in an oven at 550° C. for 10 minutes and then cooled to room temperature in a desiccator. Subsequently, the crucible was weighted (W_{crucible}) in a water-free environment. The filter with the ashes was placed on the crucible and heated progressively on a hot plate starting at room temperature up to 500° C. for a period of time of at least 1 hour. The crucible was then transferred to a furnace and heated at 800° C. for 16 h. After being cooled at room temperature in a desiccator, the crucible was weighted again (W_{crucible+ash}) in a water-free environment. AIA (%)=[(W_{crucible+ash}−W_{crucible})/Wₛₐₘₚₗₑ]×100.
D50, D90: The method of determining the particle size distributions is complying with method <429> of the United Stated Pharmacopeia (USP40), and is based on the ISO standard 13320-1. A sample powder is first poured inside a vibrating hopper to feed with a regular flow a Mastersizer 3000 (Malvern). Using an air disperser device, the powder particles were blown through a laser beam with an obscuration of the light between 1 and 15%, to reach a sufficient signal-to-noise ratio of detector and to avoid multiple scattering. The light scattered by particles at different angles is measured by a multi-element detector. The use of red and blue light, coupled to the Mie theory allows the calculation of the volumetric size distribution, where particles were considered as spheres and hence an equivalent sphere size was determined. From the obtained size distribution the cumulative volume fractions at 10, 50 and 90% were determined to give D10, D50 and D90, respectively. The median diameter D50 gives an idea of the particle size of the powder, while D10 and D90 allows to quantify finer and coarser particle sizes.
CIELAB L*, a* and b* represent the most complete colour space specified by the International Commission on Illumination (Commission Internationale d'Eclairage). It describes all the colours visible to the human eye and was created to serve as a device independent model to be used as a reference. The L* and b* values of a sample are obtained by placing the sample in a glass cell (filled about half) of a colorimeter. The used colorimeter was a Minolta CR400 Colorimeter
Determination of C₀:
Sample preparation for rheology measurements:
Reconstituted skimmed milk was used as the aqueous medium. The skimmed milk in powdered form was provided by Isigny-Ste-Mere (Isigny. France). The skimmed milk was reconstituted by dissolving powdered skimmed milk at 10% w/w in ultrapure water (18.2 MΩcm resistivity) under stirring for 4 hours at room temperature. In particular, to prepare 1000 g of reconstituted skimmed milk, 108.66 g of skimmed milk powder (DS=92.03 wt %) were dissolved in 891.34 g of ultrapure water. Dispersions of various seaweed powders were prepared in variable proportions (0.1 to 1 w/w. dry matter basis) in reconstituted skimmed milk. The seaweed powders were weighed in the suitable final proportion. thoroughly mixed with 5 wt % sucrose (to promote the rehydration) and slowly dispersed in the reconstituted skimmed milk under magnetic stirring (500 rpm). Stirring was maintained for 30 minutes at room temperature. Subsequently. the sample was heated to 80° C. for about 30 minutes under stirring at 500 rpm and held at this temperature for an additional 3 minutes.

Measurements of Storage Modulus G′:

Rheological measurements were carried out using a MCR 302 controlled-stress rheometer (Anton Paar Physica) equipped with a 50 mm plate-and-plate geometry with both upper and lower surface crosshatched. The rheometer is also equipped with a Peltier temperature controller. The gap was fixed at 1 mm Before measurements. samples were covered by a thin layer of paraffin oil on the edge of the sample to avoid evaporation during measurements. Dynamic oscillatory or viscoelastic measurements were selected to evaluate the gelation kinetics and texturizing properties of each formulated system. For these measurements, the sample was poured onto the MCR 302 plate pre-heated at 80° C. and subjected to a temperature sweep test (2° C./min) from 80° C. down to 10° C., followed by a time sweep experiment for 15 minutes at a frequency of 0.4 Hz to ensure that the system reach an equilibrium state after this considered time at 10° C. due to reorganization (structural rearrangements). Subsequently, the sample was subjected to a frequency sweep from 100 to 0.01 Hz at a constant shear strain in the linear viscoelastic region (LVE) fixed at 0.2%. To ensure that viscoelastic measurements were carried out in the LVE domain, strain sweep experiments were conducted from 0.01% to 100% at 0.4 Hz.

In all these rheological experiments. each measurement was performed at least in duplicate.

Data Processing: G′

The G′ values considered in this patent were collected from the mechanical spectra (frequency sweep test) at 0.4 Hz at 10° C. In fact as the mechanical spectra represents the real structural behavior of the obtained gels, it appeared suitable to use this G′ value as the most appropriate parameter.

Based on the G′ values obtained for all investigated samples at various concentrations, a power-law relationship (see Formula 1) was used to describe the data. Note that c* represents the lowest concentration below which there is no gel-like behavior or implicitly the critical gelling concentration. C is the seaweed powder concentration (dry matter basis); n represents the exponent value of the fitting model; k and k′ are constant factors of the fitting model

G′=k′*(C−C₀)ⁿ Formula 1

To compare samples, Formulas 2-4 were used:

G′=p*k*Cⁿ Formula 2

G′_{sample A}=k* Cⁿ Formula 3

G′_{sample B}=p* k* Cⁿ Formula 4
where p is a translational shifting factor. If p=1, that means sample A displays similar gel strength as sample B; if p>1. that means sample B displays higher G′ than Sample A; if p<1 that means sample B displays lower G′ than Sample A.

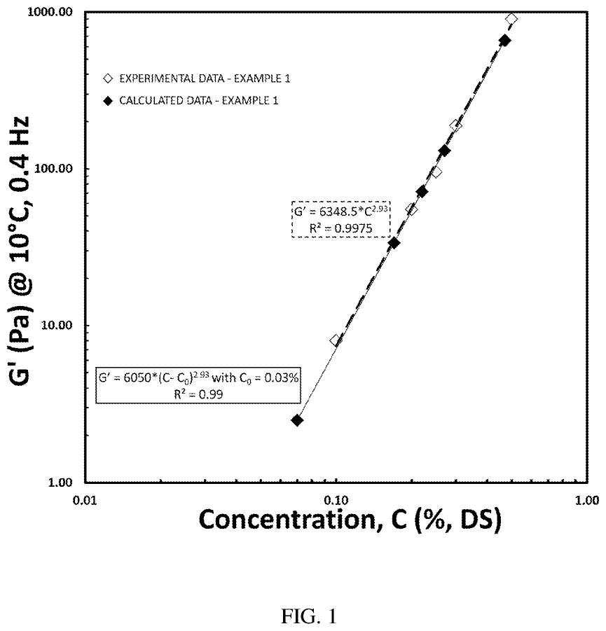

Data processing: C₀
For the determination of C₀, the following steps were respected:
(i) The storage modulus G′ values collected from the mechanical spectra as described above were plotted as a function of seaweed powder concentration, C (%, DS), in logarithmic scales (see FIG. 1).
In FIG. 1, the dashed lines and the solid lines represent the fitting of the power law formulas 3 and 1, respectively, to the experimental data (raw data) and to the estimated data. The data utilized in FIG. 1, belongs to Example 1.
(ii) Following the approach described in literature (e.g. Agoda-Tandjawa, G., Dieudé-Fauvel, E., Girault, R. & Baudez, J.-C. (2013). Chemical Engineering Journal, 228, 799-805) equation G′=kCⁿ was mathematically transformed in the form G′=k′(C−C₀)ₙ using linear regression. In this second equation, k′ represents the scaling factor, and C₀ the concentration below which no gel-like behaviour can be achieved. Note that the linear regression was performed for all investigated seaweed powders following the condition G′=kCⁿ=k′(C−C₀)ⁿ, with both exponents (n) values being identical and C>C₀.
The validation of C₀ determined using the above fitting model was verified by evaluating the rheological behaviour of all seaweed powders in similar conditions as described previously in other to evidence the gel-like behaviour.

The invention will now be described with the help of the following examples and comparative experiments, without being however limited thereto.

### Example 1: Kappaphycus alvarezii Based Powder

Fresh harvested (less than 6 h from the harvest) Kappaphycus alvarezii (Eucheuma cottonii) seaweed was rinsed with seawater and used to make a biomass having a DS of about 10 wt %. Seawater from the location of the harvest was used. The biomass was placed on a wooden table to form a biomass bed having an areal density of about 10 Kg/m². The table was placed in a sunny location and covered with a transparent tarpaulin to fully enclose it and prevent air flow. Due to the action of the sun, the temperature under the tarpaulin reached about 60° C. and a humidity over 90%. The seaweed was allowed to naturally exude in this environment for a period of time between 24 h and 72 h depending on the weather.

After exudation, the tarpaulin was removed and the biomass was kept for another 24 h in open air under the sun for drying to reach a DS of about 78 wt %.

The dried biomass was subsequently placed in volume of tap water sufficient to cover the seaweed entirely and the seaweed was allowed to rehydrate for 1 h at room temperature without stirring. The rehydrated seaweed was then collected using a filter and a biomass having a DS around 40 wt % was obtained.

The biomass containing the rehydrated seaweed was cooked in brine solution (100 g/L of KCl) at 90° C. for 30 minutes. The weight of the brine solution used for cooking was about 6 times the mass of the seaweed. After cooking, the brine solution was drained and the recovered seaweed was washed by placing it in a volume of tap water at room temperature for 10 minutes. Enough water was used to completely cover the seaweed.

The seaweed was then collected using a filter and dried using a belt dryer for 30 minutes at 60° C. and led to a final product of about 94.9% DS. The dried product was milled into a powder with a Retsch mill (final sieve at 0.25 mm). The properties of the obtained seaweed powder are given in Table 1:

TABLE 1

| Property | | Seaweed powder |
|---|---|---|
| Chloride (Cl⁻) (%) | | 4.4 |
| AIM (%) | | 11.20 |
| AIA (%) | | 0.3 |
| Color | L* | 78.4 |
| | a* | 1.3 |
| | b* | 14.2 |
| Particle size | D50 (μm) | 86.8 |
| | D90 (μm) | 228 |
| | D10 (μm) | 16 |
| | D [4.3] | 107 |
| | Span | 2.432 |

| | |
|---|---|
| Cₒ × 10⁻² (% DM) | 4.0 |
| G′ (Pa) | 130 |

### Example 2: Chondrus crispus Seaweed Powder

A fresh Chondrus crispus was harvested from the wild. It was processed like in Example 1 and was kept between 3 and 72 hours under a tarpaulin. In some instances, the seaweed was turned over during the exudation to allow a homogeneous exposure to sunlight. The seaweed was then sun dried over a period ranging from 1 to 3.5 days, depending on the weather, to reach a DS of about 65 wt % (approximatively 35 wt % moisture). The seaweed was further processed as in Example 1.

The dried biomass was subsequently placed in volume of tap water sufficient to cover the seaweed entirely and the seaweed was allowed to rehydrate for 1 h at room temperature without stirring. The rehydrated seaweed was then collected using a filter and a biomass having a DS around 40 wt % was obtained.

The biomass containing the rehydrated seaweed was cooked twice in brine solution (350 g/L of KCl) at 90° C. for 30 minutes. The weight of the brine solution used for cooking was about 16 times the mass of the seaweed. After cooking, the brine solution was drained and the recovered seaweed was washed by placing it in a volume of tap water at room temperature for 10 minutes. Enough water was used to completely cover the seaweed.

The seaweed was then collected using a filter and dried using a belt dryer for 30 minutes at 60° C. and led to a final product of about 94.3% DS. The dried product was milled into a powder with a Retsch mill (final sieve at 0.25 mm) and sieved at 0.25 mm.

The properties of the obtained seaweed powder are given in Table 2:

TABLE 2

| Property | | Seaweed powder |
|---|---|---|
| Chloride (Cl⁻) (%) | | 0.1 |
| AIM (%) | | 8.1 |
| AIA (%) | | 0.1 |
| Color | L* | 58.9 |
| | a* | 5.3 |
| | b* | 8.9 |
| Particle size | D50 (μm) | 164 |
| | D90 (μm) | 310 |
| | D10 (μm) | 29 |
| | D [4.3] | 171 |
| | Span | 1.710 |

| | |
|---|---|
| Cₒ × 10⁻² (% DM) | 3.5 |
| G′ (Pa) | 140 |

### Example 3: Various Seaweeds

Fresh harvested (less than 6 h from the harvest) seaweeds were kept for 24 h in open air under the sun for drying to reach a DS between 60 and 95 wt %.

The seaweeds were then further dried in an oven at 60° C. overnight.

The dried seaweeds were milled into a powder with a Retsch mill (final sieve at 0.25 mm) and sieved at 0.25 mm. The properties of the obtained powders are given in Table 3.

TABLE 3

| Property | | Cottonii | Spinosum | Chondrus |
|---|---|---|---|---|
| Chloride (Cl⁻) (%) | | 21.8 | 12.9 | 0.7 |
| AIM (%) | | 11.2 | 8.4 | 9.3 |
| AIA (%) | | 0.2 | 0.8 | 0.1 |
| Color | L* | 67.5 | 80.1 | 64.0 |
| | a* | 2.6 | 1.0 | 3.4 |
| | b* | 8.1 | 16.9 | 14.8 |
| Particle size | D50 (μm) | 117 | 111 | 171 |
| | D90 (μm) | 276 | 258 | 299 |
| | D10 (μm) | 19.7 | 18 | 43 |
| | D [4.3] | 133 | 125 | 175 |
| | Span | 2.191 | 2.162 | 1.498 |

| | | | |
|---|---|---|---|
| Cₒ × 10⁻² (% DM) | 6.4 | 16.0 | 19.0 |
| G′ [Pa] | 28 | 21 | 15 |

### Example 4

The Kappaphycus alvarezii (Cottonii) powder of Example 1 and locust bean gum (LBG) compositions were prepared at a total seaweed and LBG concentration of 1 w/w % in variable proportions (25/75, 40/60, 50/50, 60/40, 75/25, 90/10, 95/5 and 97.5/2.5) in distilled water. For sample preparation, locust bean gum powder and seaweed powder were weighed in a suitable final proportion, thoroughly mixed and slowly dispersed in water under magnetic stirring (500 rpm). Stirring was maintained for 30 minutes at room temperature to ensure a good dispersion. Then, the sample was heated to 80° C. (for 30 minutes) under stirring at 500 rpm.

### Rheological Characterization and Results of Example 4

Rheology Measurements
Rheological measurements were carried out using a MCR 301 controlled-stress rheometer (Anton Paar Physica) equipped with a Couette device. The rheometer was also equipped with a Peltier temperature controller. Before measurements, samples were covered by a thin layer of paraffin oil to avoid evaporation during measurements. Dynamic oscillatory or viscoelastic measurements were selected to evaluate the gelation kinetics and texturizing properties of each investigated composition. For these measurements, sample was poured onto the MCR 302 plate pre-heated at 80° C. and was subjected to a temperature sweep test (2° C./min) from 80° C. down to 10° C., followed by a time sweep experiment for 15 minutes at a frequency of 0.4 Hz to ensure that the system reached an equilibrium state (structural rearrangements). After that, sample was subjected to a frequency sweep, from 100 to 0.01 Hz at a constant shear strain in the linear viscoelastic region (LVE), fixed at 0.3%. To ensure that viscoelastic measurements were carried out in the LVE domain, strain sweep experiments were conducted from 0.01% to 100% at 0.4 Hz. In all these rheological experiments, each measurement was performed at least in duplicate, from new sample preparations. The storage modulus (G′) values collected from the mechanical spectra at 0.1 Hz and at 10° C. are used for the comparison of all investigated samples.

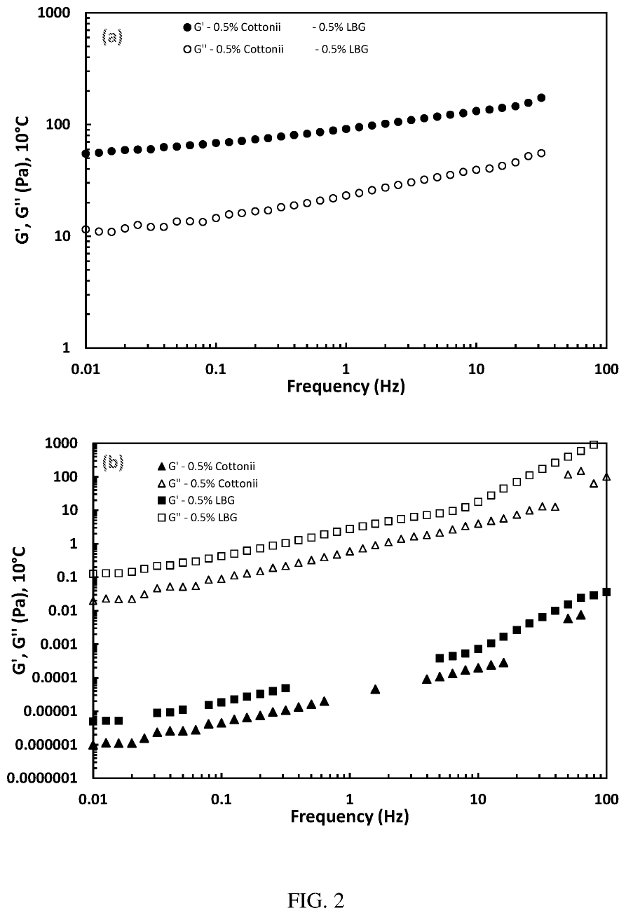

FIG. 2 demonstrate that blending Kappaphycus alvarezii powder and locust bean gum in distilled water, at appropriate total concentration and mixing ratio, gives a synergistic gel although each component taken alone did not form a gel at experimental conditions. Indeed, the gel formation of the inventive composition induced by synergistic interaction is evidenced by G′>10G″ and relatively frequency-independent moduli (FIG. 2a), whereas the rheological behaviour of the individual components is that typical of liquid viscoelastic character with G″>G′ over a wide frequency range (FIG. 2b).

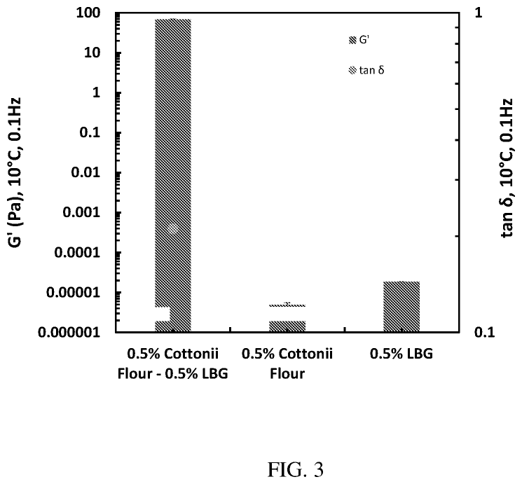

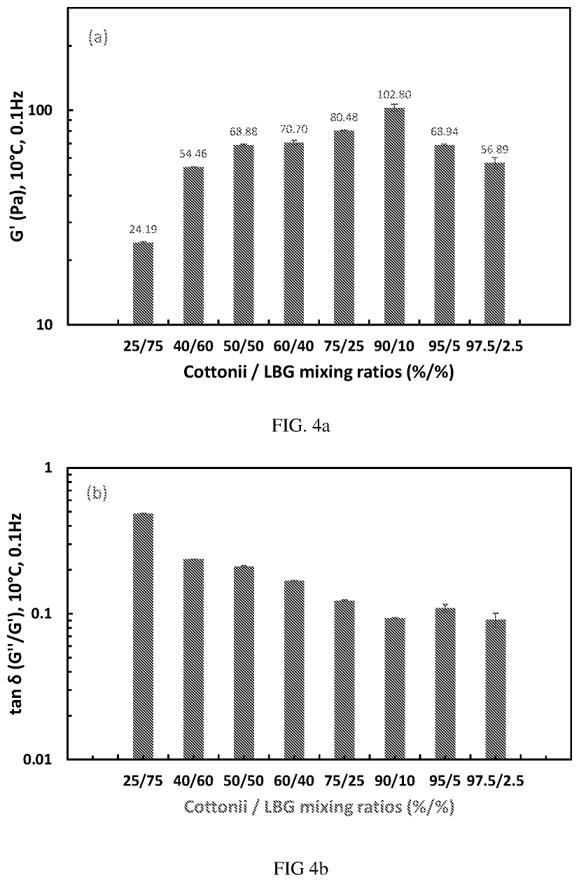

The storage modulus (G′) representing the gel strength of the resulting blend of Kappaphycus alvarezii powder and locust bean gum (50/50) is 68.88 Pa±0.64, whereas negligible values are obtained for individual LBG and Kappaphycus alvarezii powder viscous solutions (G′<<10⁻⁴ Pa) (FIG. 3). While varying the mixing ratio of Kappaphycus alvarezii/locust bean gum from 25/75 to 97.5/2.5, the synergistic gels exhibited an increase of their viscoelastic properties with the increase of Kappaphycus alvarezii content in the mixed systems from 25% w/w to 90% w/w at which they reach an optimal synergy (G′=103 Pa±0.64) and then decreased with further increase of the Kappaphycus alvarezii content from 90% w/w to 97.5% w/w (FIG. 4a). Additionally, the lower LBG content in the mixed system, the more structured or well-organized network structure is obtained for the synergistic gels, in respect to the experimental conditions (FIG. 4b).

### Examples 5 and 6

Kappaphycus alvarezii or Eucheuma spinosum powders were mixed with a native waxy corn starch (sold by Cargill as SimPure™ 99400) or with a native potato starch (sold by Cargill as SimPure™ 99500) in variable proportions in distilled water in the presence of 0.3% KCl (when Kappaphycus alvarezii was used) or 1.5% NaCl (when Eucheuma spinosum was used). Seaweed powder and starch concentrations in the mixtures were, respectively at 0.30% w/w and 2% w/w. For sample preparation, starch and seaweed powder were weighed in a suitable final proportion, thoroughly mixed and slowly dispersed in water under magnetic stirring (500 rpm). Stirring was maintained for 30 minutes at room temperature to ensure seaweed powder homogeneous dispersion and complete hydration of starch granules. Then, the sample still under stirring at 500 rpm was heated to 80° C. (for SimPure™ 99500-based samples) or 95° C. (for SimPure™ 99400-based samples) for—30 minutes (in both cases), and held at this temperature for supplemental 10 minutes. The same sample preparation methods were rigorously used to prepare each seaweed powder dispersions (0.30% w/w) and starch suspensions (2% w/w) in distilled water.

### Rheological Characterization and Results of Examples 5 and 6

Rheology Measurements
Rheological measurements were carried out using a MCR 302 controlled-stress rheometer (Anton Paar Physica) equipped with a 50 mm plate-and-plate geometry with both upper and lower surface crosshatched. The rheometer was also equipped with a Peltier temperature controller. Before measurements, samples were covered by a thin layer of paraffin oil on the edge of the sample to avoid evaporation during measurements, with a gap fixed at 1 mm Dynamic oscillatory or viscoelastic measurements were selected to evaluate the gelation kinetics and texturizing properties of each formulated composition. For these measurements, a sample poured onto the MCR 302 plate pre-heated at 80° C. was subjected to a temperature sweep test (2° C./min) from 80° C. down to 10° C., followed by a time sweep experiment for 15 minutes at a frequency of 0.4 Hz to ensure that the system reached an equilibrium state (structural rearrangements). After that, the sample was subjected to a frequency sweep, from 100 to 0.01 Hz at a constant shear strain in the linear viscoelastic region (LVE), fixed at 0.3%. To ensure that viscoelastic measurements were carried out in the LVE domain, strain sweep experiments were conducted from 0.01% to 100% at 0.4 Hz. In all these rheological experiments, each measurement was performed at least in duplicate, from new sample preparations.
The storage modulus (G′) values collected from the mechanical spectra at 0.1 Hz and at 10° C. were used for the comparison of all investigated samples. Additionally, the synergistic effect of seaweed powder/starch composition was determined by calculating the rheological synergism (R) as followed:

$$R = \frac{{G}_{mixture}^{′} - ( {G}_{starch}^{′} + {G}_{seaweed}^{′} )}{{G}_{starch}^{′} + {G}_{seaweed}^{′}}$$

Where G′ₘᵢₓₜᵤᵣₑ represents the G′value obtained for the composition, G′_{starch} and G′_{seaweed} were the values obtained for the individual starch suspension and seaweed powder, respectively.

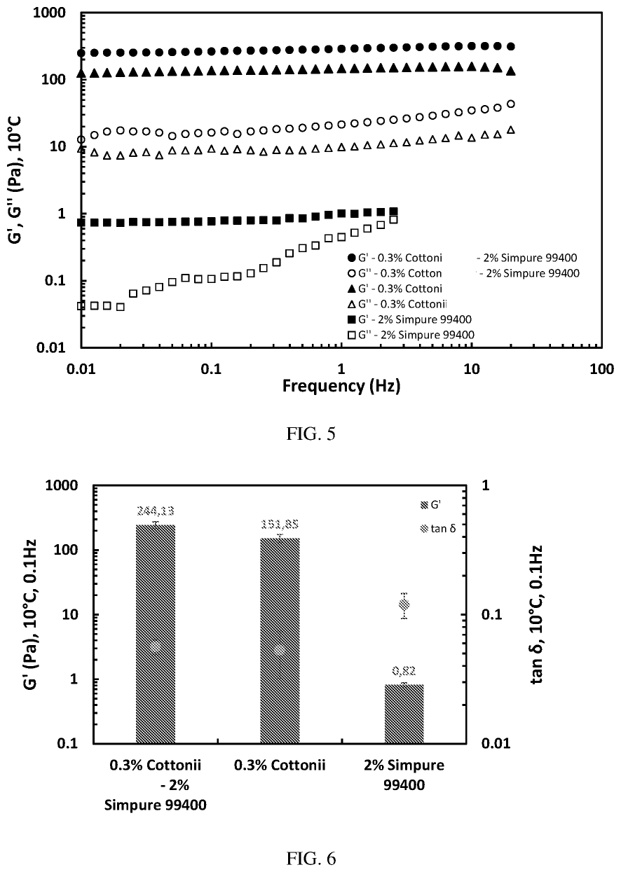

FIG. 5 demonstrates that the Kappaphycus alvarezii powder/Simpure 99400 starch mixture displayed a typical true gel-like behavior with G′>10G″ and relatively frequency-independent moduli as for pure Kappaphycus alvarezii powder, in respect to the defined medium condition. For the starch granules in suspension in distilled water, a particular very weak gel-like behavior was observed with G′>G″ at low frequency and G′ displaying a plateau at this frequency range. Swollen starch granules filled in the Kappaphycus alvarezii gel network led to improved viscoelastic properties in comparison to pure Kappaphycus alvarezii powder (FIG. 6). Similar results were obtained for the composition containing Simpure 99500.

Clearly, the R value obtained as described above is positive (R=0.60) that is an evidence of a real synergistic effect, leading to an improved gel strength (FIGS. 5 and 6).

### Examples 7 Vegan Milk with Eucheuma spinosum Powder/LBG Composition

A commercially available almond milk containing inter alia 0.042% gellan gum and water was used as control. The gellan gum was replaced by a Eucheuma spinosum powder/LBG composition in the ratios presented in Table 3.

TABLE 3

| | | 1:1 | 20% | 30% |
|---|---|---|---|---|
| ′ | Control | Replacement | Increase | Increase |
| Gellan Gum | 0.042% | 0.00% | 0.00% | 0.00% |
| LBG | 0.069% | 0.069% | 0.069% | 0.069% |
| Spinosum | 0.00% | 0.042% | 0.050% | 0.055% |

No separation was observed for the samples containing Eucheuma spinosum powder/LBG composition. The samples had a good mouthfeel with no gelation even after 18 days.

1. A seaweed-based composition comprising water, a seaweed powder and an additional component, said additional component being chosen from the group consisting of glucomannans, galactomannans, native starch and combinations thereof, wherein the seaweed comprises a red seaweed.

2. The seaweed-based composition according to claim 1, wherein the additional component is chosen from the group consisting of guar gum, xanthan gum, locust bean gum, cassia gum, tara gum, konjac gum, alginate, agar, carrageenan, beta 1,3 glucan, native starch and combinations thereof.

3. The seaweed-based composition according to claim 1, wherein the additional component is used in an amount of at least 1.5 wt % based on the total dry solids content of the composition.

4. The seaweed-based composition according to claim 1, wherein the water is present in the composition in an amount of at least 5 wt % based on the total weight of the composition.

5. (canceled)

6. The seaweed-based composition according to claim 1, wherein the seaweed is belonging to Rhodophyta phylum.

7. The seaweed-based composition according to claim 1, wherein the seaweed is a red seaweed selected from the families of Gigartinaceae, Bangiophyceae, Palmariaceae, Hypneaceae, Cystocloniaceae, Solieriaceae, Phyllophoraceae and Furcellariaceae or combinations thereof.

8. The seaweed-based composition according to claim 1, wherein the seaweed powder contains seaweed particles, said particles having a D50 of at least 20 μm and preferably at most 750 μm.

9. The seaweed-based composition according to claim 1, wherein the seaweed powder contains seaweed particles, said particles having a D90 of at least 125 μm.

10. A food product, a beverage, a nutritional product, a dietary supplement, a feed product, a personal care product, a pharmaceutical product or industrial product comprising the seaweed-based composition according to claim 1.

11. The seaweed-based composition according to claim 8, wherein said particles have a D50 of at most 750 μm.

12. The seaweed-based composition according to claim 9, wherein said particles have a D90 of at most 800 μm.

13. The seaweed-based composition according to claim 1, wherein the seaweed powder has a storage modulus (G′) of 10 Pa to 500 Pa.

14. The seaweed-based composition according to claim 1, wherein the seaweed powder has a critical gelling concentration (Co) of 0.001 wt % to 0.5 wt %.

15. The seaweed-based composition according to claim 1, wherein the seaweed powder comprises 1 wt % to 50 wt % of acid insoluble material.

16. The seaweed-based composition according to claim 1, wherein the seaweed powder has a CIELAB L* value of at least 50.

17. The seaweed-based composition according to claim 1, wherein the seaweed powder has a CIELAB a* value of at most 5.0.

18. The seaweed-based composition according to claim 1, wherein the seaweed powder has a CIELAB b* value of at most 20.
